(19) United States
(12) Patent Application Publication
(10) Pub. No.: US 2018/0315503 A1
(43) Pub. Date: Nov. 1, 2018

(54) MIND-ALTERING SUBSTANCE TESTING SYSTEM

(71) Applicant: Alcolizer Pty Ltd, Cleveland (AU)

(72) Inventors: Roger HUNT, Cleveland (AU); James BROWN, Cleveland (AU)

(21) Appl. No.: 15/770,303

(22) PCT Filed: Oct. 24, 2016
(86) PCT No.: PCT/AU2016/050996

§ 371 Date: Apr. 23, 2018

(30) Foreign Application Priority Data
Oct. 23, 2015 (AU) 2015904346

(51) Int. Cl. G16H 40/67; A61B 5/00

(52) U.S. Cl. CPC G16H 40/67; A61B 5/0022; A61B 5/746; A61B 5/4845

(57) ABSTRACT

The present invention relates to an online mind-altering substance testing system. The system includes distributed mind-altering substance testing units for performing mind-altering substance tests. A server is provided for receiving results of the mind-altering substance tests. A database is provided for storing the results. A display is provided for displaying the stored results, preferably in a dashboard of graphs to facilitate rapid and intuitive evaluation of often large numbers of tests from various testing units. Advantageously, a reviewer may review live stored results from multiple testing units once the tests have been performed, which enables rapid risk assessment and corrective action to be undertaken. For example, the effect of failed tests may be more frequent at a certain site prompting a need for investigating a cause at that site.

### TECHNICAL FIELD

The present invention generally relates to a mind-altering substance testing system used in testing for the presence of mind-altering substances, such as alcohol or drugs.

### BACKGROUND

The reference to any prior art in this specification is not, and should not be taken as an acknowledgement or any form of suggestion that the prior art forms part of the common general knowledge.

Mind-altering substances may impair human performance. Employers can test their employees for mind-altering substances such as alcohol or drugs with a view of ensuring optimum results and compliance in the workplace.

Some workplaces are fitted with wall mounted alcohol breath testers. Workers may be required to pass the test prior to commencing work. Other workplaces conduct random breath tests of employees using hand held breath test units. Such tests are focused on the individual and it is straightforward to determine whether an individual has an unacceptable Blood Alcohol Concentration. However, the Applicant has perceived that it can often be difficult to determine from individual failures, which are often poorly documented, whether the company has a wider problem with compliance.

The preferred embodiment provides an improved system for evaluating company-wide mind-altering substance testing.

### SUMMARY OF THE INVENTION

According to one aspect of the present invention, there is provided an online mind-altering substance testing system including:
distributed mind-altering substance testing units for performing mind-altering substance tests;
a server for receiving results of the mind-altering substance tests;
a database for storing the results; and
a display for displaying the stored results.

Advantageously, a reviewer may review live stored results from multiple entities once the tests have been performed, which enables rapid risk assessment and corrective action to be undertaken. For example, the effect of failed tests may be more frequent at a certain site prompting a need for investigating a cause at that site.

Preferably, the stored results are displayed collectively in a dashboard, in turn, including one or more graphs relating to the stored results. Advantageously, the graphs facilitate rapid and intuitive evaluation of often large numbers of tests from various entities (e.g. companies, divisions, sites, etc). The dashboard may enable drilling down into displayed test data to obtain a particular test result.

The graphs may be arranged in accordance with alcohol testing or drug testing. The dashboard may include filter selectors which can be selected to filter underlying stored test data so that the graphs are modified accordingly.

The system may perform diagnostics on the testing units. The dashboard may display any alerts based on the diagnostics. The displayed alerts may include warning alerts or error alerts. The alerts may be selectable to produce further details.

One or more of the graphs may be grouped in accordance with different parameters. The graphs may be selectable to produce a group of itemized tests. The group may be filtered or sorted using parameters. Each test in the group may be selected to display an associated test result. Each test result may be either an alcohol result, or a drug screen and toxicology result.

The system may be configured to amend parameters relating any one or more of: a company, divisions of a company, sites, staff details, user details, system settings and testing units. The system may be configured to modify, either globally or individually, operational configuration settings of one or more of the testing units.

The system may be configured to manage collection of samples and approval of tests. The system may include a sample repository in which test samples are provided. The system may be configured to synchronize sample statuses with another server. The system may be configured to generate reports including the dashboard or test data.

Prior to accessing the dashboard or any other test data, the system may validate a user using a SMS code. The validation may also involve using a user name and password

The testing units may be wall mounted units or handheld units.

According to another aspect of the present invention, there is provided an online mind-altering substance testing method including:
performing mind-altering substance tests using distributed mind-altering substance testing units;
receiving the results of the mind-altering substance tests;
storing the received results; and
displaying the stored results.

According to another aspect of the present invention, there is provided an online mind-altering substance testing tool, the tool including a display for displaying stored results of mind-altering substance tests, the tests being performed by distributed mind-altering substance testing units.

Any of the features described herein can be combined in any combination with any one or more of the other features described herein within the scope of the invention.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a block diagram of an online mind-altering substance testing system in accordance with an embodiment of the present invention;

FIG. 2 is a schematic diagram of a graphical dashboard of the system of FIG. 1;

FIG. 3 is a expanded list of itemized tests produced when selecting a graph of the dashboard of FIG. 2; and

FIG. 4 is an example test report displayed when selecting one of the items in the list of tests of FIG. 3.

### DETAILED DESCRIPTION OF PREFERRED EMBODIMENTS

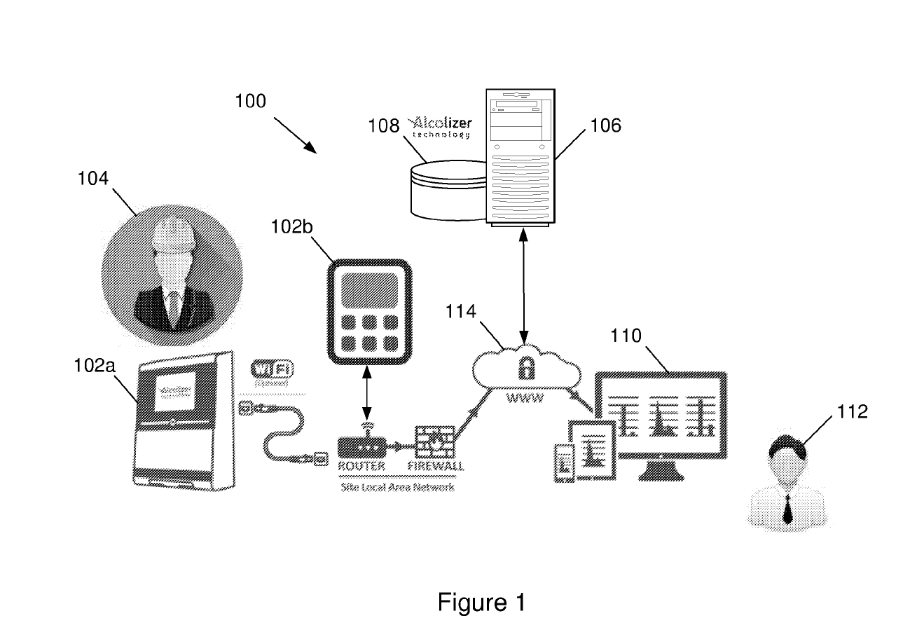

According to an embodiment of the present invention, there is provided an online mind-altering substance testing system 100 as shown in FIG. 1. The system 100 includes mind-altering substance testing units 102a, 102b for performing mind-altering substance tests on employees 104. The testing units 102 are typically wall mounted units 102a or handheld units 102b, and are distributed throughout many companies and sites.

An administration server 106 receives results of the mind-altering substance tests, and stores the results in a database 108. The system 100 further includes a company computer 110, with electronic display, enabling a company manager 112 to review the stored results which are displayed. In practice, many company computers 110 from different companies would be provided to review their employee test results. The electronic testing units 102, administration server 106 and company computer 110 are all coupled together in two-way communication over the Internet 114.

Advantageously, the manager 112 (i.e. reviewer) can review the live stored results from multiple entities once the tests have been performed, which enables rapid corrective action to be taken by the company. For example, the effect of failed tests may be more frequent at a certain worksite prompting a need for investigating a cause at that site.

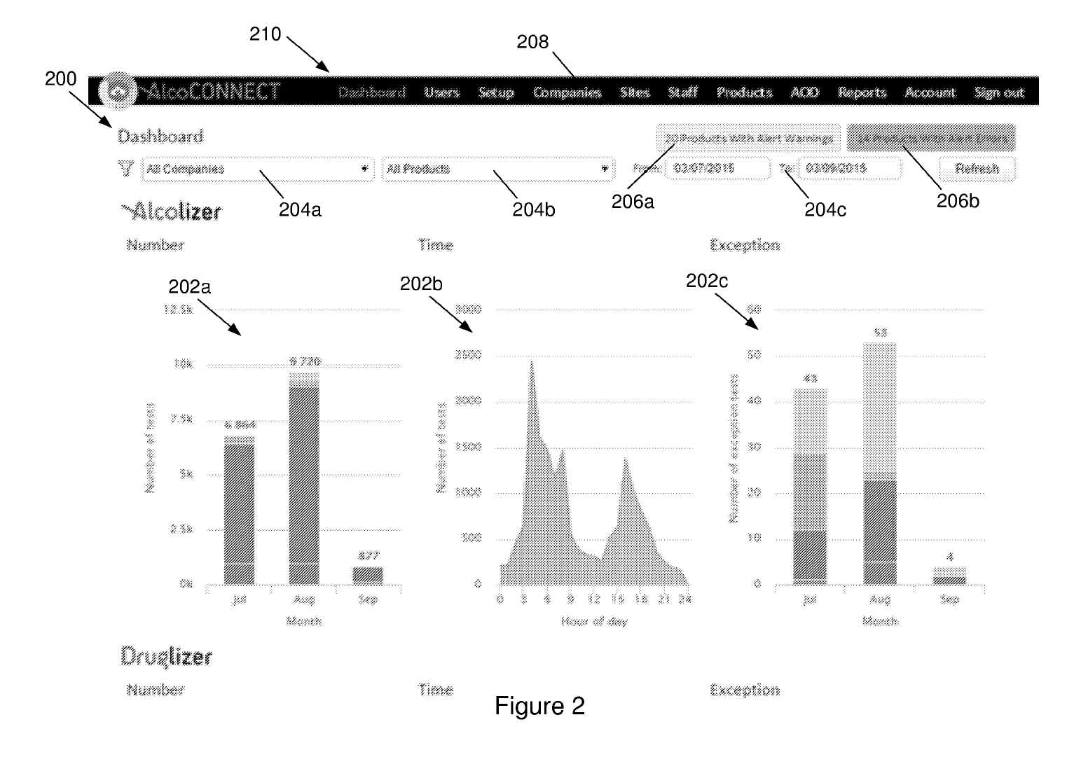

Turning to FIG. 2, the management computer 110 includes a dashboard 200 displaying graphs 202a, 202b, 202c of the collective stored results. Advantageously, the graphs 202 facilitate rapid and intuitive evaluation of often large numbers of tests from various entities (e.g. companies, divisions, sites, etc). The manager 112 can easily review and analyse stored test data by number of tests, site location, time of day, result and employee 104. The dashboard 200 enables drilling down into displayed test data to obtain a particular test result.

The displayed graphs 202 are arranged in accordance with alcohol testing at the top of the dashboard 200, and analogous drug testing displayed underneath (not shown). The dashboard 200 includes company, testing unit (product) and date range filter selectors 204a, 204b 204c which can be used to filter the underlying stored test data so that the graphs 202a, 202b, 202c are automatically modified accordingly.

The system 100 performs diagnostics on the testing units 102. The dashboard 200 displays any alerts 206 based on the diagnostics. The displayed alerts 206 include warning alerts 206a indicative of units 102 being close to failing tests or error alerts 206b indicative of units 102 failing tests. Selecting either alert 206 produces an expanded list of itemized alerts which, in turn, can be selected to provide detailed individual alert reports.

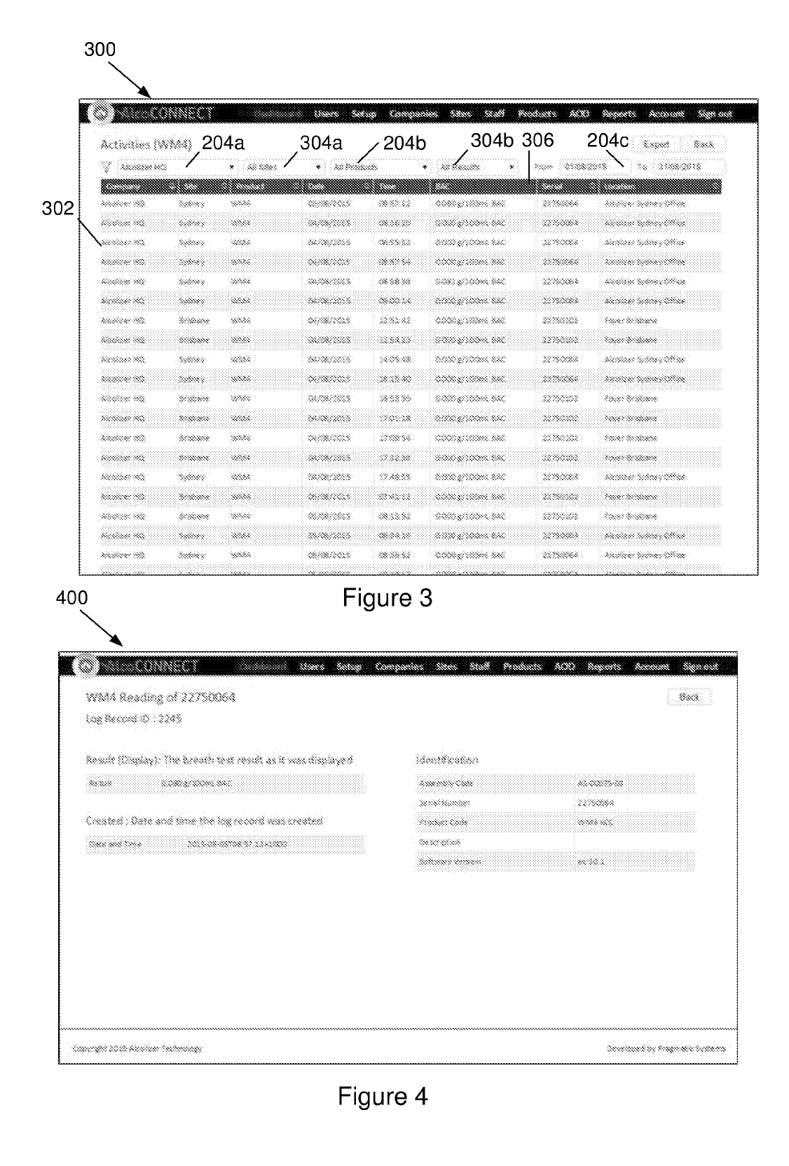

The displayed graphs 202 include number of tests over time 202a, tests over time of day in which the tests were taken 202b, and exception (i.e. failed) tests over time 202c. The graphs 202a, 202c are vertically grouped (by colored bands) in columns by company, or test type if filter selector 204a is set to a particular company. Selecting the graphs 202 produces an expanded list 300 of itemized tests 302 shown for example in FIG. 3. The list 300 can be filtered using previous filters 204a, 204b 204c, as well as a site filter 304a and result filter 304b. The list 300 can also be sorted by selecting headings 306.

Each test 302 in the resulting list 300 can be selected to provide detailed individual test results 400 shown for example in FIG. 4. Each test result 400 can be either an alcohol result, or a drug screen and toxicology result. Each stored test in the database 108 is associated with the employee 104, company, division, site and testing unit 102.

Returning to FIG. 2, the manager 112 can select displayed menu items 208 from the top menu 210 to configure or display system parameters. The menu items 208 can be selected to amend parameters relating to a company and associated divisions, a division and associated sites, system settings, details of staff 104 or users 112, or testing units 102 (i.e. products). For example, the operational configuration settings of one or more of the testing units 102 can be modified, either globally or individually using the “products” menu item. The “AOD” menu item 208 in the top menu 210 can be used to manage collection of samples and approval of drug tests with mobile equipment or third party suppliers or laboratories (e.g. Hansaworld). The system 100 includes a sample repository in which drug test samples are provided, and is configured to synchronize sample statuses with a laboratory server (not shown). The “reports” menu item 208 in the top menu 210 can be used to generate reports including the dashboard 200 or test data shown in FIGS. 3 and 4, for example.

The security of the sensitive tests stored in the database 108 must be maintained. Accordingly, prior to accessing the dashboard 200 or any other test data, the manager 112 must be validated using a SMS code in addition to a conventional login (user name and password). Logging in involves the manager 112: entering a username and password into the computer 110, receiving a mobile phone SMS code responsive to entering a valid username and password, and then entering the SMS code into the computer 110 to validate the manager 112.

A person skilled in the art will appreciate that many embodiments and variations can be made without departing from the ambit of the present invention.

In one embodiment, the server 106 and computer 110 may include the same computational device.

In compliance with the statute, the invention has been described in language more or less specific to structural or methodical features. It is to be understood that the invention is not limited to specific features shown or described since the means herein described comprises preferred forms of putting the invention into effect.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

1. An online mind-altering substance testing system including:
distributed mind-altering substance testing units for performing mind-altering substance tests;
a server for receiving results of the mind-altering substance tests;
a database for storing the results; and
a display for displaying the stored results.

2. A testing system as claimed in claim 1, wherein a reviewer can review live stored results from multiple testing units once the tests have been performed, which enables rapid risk assessment and corrective action to be undertaken.

3. A testing system as claimed in claim 1, wherein the stored results are displayed collectively in a dashboard, in turn, including one or more graphs relating to the stored results, the graphs facilitating rapid and intuitive evaluation of often large numbers of tests from various testing units, the dashboard enabling drilling down into displayed test data to obtain a particular test result, and the graphs being arranged in accordance with alcohol testing or drug testing, the dashboard including filter selectors which can be selected to filter underlying stored test data so that the graphs are modified accordingly.

4-6. (canceled)

7. A testing system as claimed in claim 1, wherein the system performs diagnostics on the testing units, and a dashboard displays any alerts based on the diagnostics, the displayed alerts including warning alerts or error alerts.

8-9. (canceled)

10. A testing system as claimed in claim 7, wherein the alerts are selectable to produce further details.

11. A testing system as claimed in claim 1, wherein the stored results are displayed collectively in a dashboard, in turn, including one or more graphs relating to the stored results, the graphs facilitating rapid and intuitive evaluation of often large numbers of tests from various testing units, one or more of the graphs being grouped in accordance with different parameters.

12. A testing system as claimed in claim 11, wherein the graphs are selectable to produce a group of itemized tests.

13. A testing system as claimed in claim 12, wherein the group can be filtered or sorted using parameters.

14. A testing system as claimed in claim 12, wherein each test in the group can be selected to display an associated test result.

15. A testing system as claimed in claim 14, wherein each test result is either an alcohol result, or a drug screen and toxicology result.

16. A testing system as claimed in claim 1, configured to amend parameters relating any one or more of: a company, divisions of a company, sites, staff details, user details, system settings and testing units.

17. A testing system as claimed in claim 1, configured to modify, either globally or individually, operational configuration settings of one or more of the testing units.

18. A testing system as claimed in claim 1, configured to manage collection of samples and approval of tests.

19. A testing system as claimed in claim 18, including a sample repository in which test samples are provided.

20. A testing system as claimed in claim 1, configured to synchronize sample statuses with another server.

21. A testing system as claimed in claim 1, configured to generate reports including dashboard or test data.

22. A testing system as claimed in claim 1 which validates a user using a SMS code and/or a user name and password.

23. A testing system as claimed in claim 1, wherein the testing units are wall mounted units or handheld units.

24. An online mind-altering substance testing method including:
performing mind-altering substance tests using distributed mind-altering substance testing units;
receiving the results of the mind-altering substance tests;
storing the received results; and
displaying the stored results.

25. An online mind-altering substance testing tool, the tool including a display for displaying stored results of mind-altering substance tests, the tests being performed by distributed mind-altering substance testing units.
